(19) United States
(12) Patent Application Publication
(10) Pub. No.: US 2015/0352165 A1
(43) Pub. Date: Dec. 10, 2015

(54) CLINDAMYCIN PHOSPHATE, SALICYLIC ACID AND TEA TREE OIL COMBINATIONS

(71) Applicant: IMUNEKS FARM MAC SANAYI VE TICARET A.S., Istanbul (TR)

(72) Inventors: Ibrahim Mustafa Iskender PISAK, Istanbul (TR); Mehmet Nevzat PISAK, Istanbul (TR); Mehmet Levent SELAMOGLU, Istanbul (TR); Semra BINGOL, Istanbul (TR)

(21) Appl. No.: 14/437,175

(22) PCT Filed: Sep. 6, 2013
(86) PCT No.: PCT/TR2013/000288

§ 371 Date: Apr. 20, 2015

(30) Foreign Application Priority Data
Sep. 11, 2012 (TR) 2012/10369

(51) Int. Cl. A61K 36/61; A61K 31/7056; A61K 47/34; A61K 47/32; A61K 47/18; A61K 47/10; A61K 31/60; A61K 9/00

(52) U.S. Cl. CPC A61K 36/61; A61K 31/60; A61K 31/7056; A61K 9/0014; A61K 47/32; A61K 47/18; A61K 47/10; A61K 47/34

(57) ABSTRACT

The present invention relates to the topical compositions comprising clindamycin phosphate, salicylic acid and tea tree oil; to the preparation methods thereof; and to the use thereof in the treatment of skin infections and acne vulgaris accompanied by inflammatory lesions.

### FIELD OF THE INVENTION

The present invention relates to the topical compositions comprising clindamycin phosphate, salicylic acid and tea tree oil; and to the preparation methods thereof. Topical compositions of the invention are used in the treatment of skin infections and acne vulgaris accompanied by inflammatory lesions.

### BACKGROUND OF THE INVENTION

The present invention relates to the topical compositions comprising clindamycin phosphate, salicylic acid and tea tree oil; to the preparation methods thereof; and to the use thereof in the treatment of skin infections and acne vulgaris accompanied by inflammatory lesions.

Clindamycin is a semi-synthetic antibiotic prepared from lincomycin. The chemical name of clindamycin is methyl 7-chloro-6,7,8-trideoxy-6-[[[(2S,4R)-1-methyl-4-propyl-2-pyrrolidinyl]carbonyl]amino]-1-thio-L-threo-α-D-galacto-octopyranoside. Its chemical structure is shown in Formula 1.

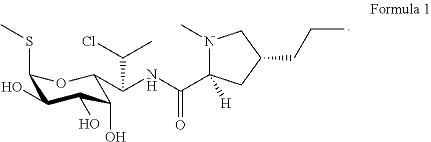

Some systemic absorption of clindamycin does occur, particularly when large surface areas of skin are treated. Systemic absorption of clindamycin phosphate is lower than that of hydrochloride salt. Therefore, phosphate salt is preferred in the topical preparations. Clindamycin is commonly used in anti-acne treatments. Gel, cream, solution, lotion and soap forms used for this purpose are available.

Salicylic acid is a keratolytic drug. The chemical name of salicylic acid is 2-hydroxybenzoic acid. Its chemical structure is shown in Formula 2.

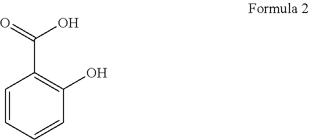

Salicylic acid is commonly used in anti-acne treatments. Furthermore, it is used for the treatment of psoriasis, ichthyosis, seborrheic dermatitis and dandruff characterized by flaking skin and hyperkeratosis. Gel, cream, solution, lotion, ointment, soap and shampoo forms used for this purpose are available.

Tea tree oil is a natural antiseptic extracted from Australian tea tree or alias Indian bay tree. It is used against acne, fungal infections of skin and nail, muscle pain, soft tissue rheumatism, hair root infection, carbuncle, wart, herpes, vulva and vaginal infections. Gel, cream, solution, lotion, soap and shampoo forms used for this purpose are available.

Skin infections are usually caused by bacteria, virus and fungus. Active agents such as topical antimicrobial agents, antiseptics and keratolytics alone or in combination are commonly used in the treatment of skin infections (Aly R. “Microbial Infections of Skin and Nails”. In: Baron S, editor. Medical Microbiology. 4th edition. Galveston (Tex.): University of Texas Medical Branch at Galveston; 1996. Chapter 98). Antimicrobial agents include antibiotics commonly used in both systemic and topical treatment of skin infections due to their antiseptic, bacteriostatic, anti-inflammatory and immunoregulatory characteristics. Topical treatment is sufficient in localized infections of outer layer of skin. Antibiotics such as clindamycin, erythromycin, mupirocin and fusidic acid are preferred as topical treatment. Another antimicrobial agent that can be used in addition to topical antibiotic treatment is an antiseptic, tea tree oil, with its broad-spectrum antibacterial, antiviral, antifungal, anti-inflammatory and immunoregulatory characteristics. Keratolytics help easily remove the dry and crusty skin by softening the keratin, a component of the skin. When combined with the other drugs, keratolytics provide effective penetration of the additional drugs by removing the outer layer of skin. Salicylic acid heads a list of keratolytics commonly used in the treatment of skin infections.

Acne vulgaris is a common skin disease that affects 85% to 100% of people at some time during their lives. The pathogenesis of acne vulgaris is multifactorial. Four key factors are responsible for the development of an acne lesion. These factors are follicular epidermal hyperproliferation with subsequent plugging of the follicle, excess sebum, the presence and activity of Propionibacterium acnes (pathogenic bacteria), and inflammation (NilFroushzadeh, M. A. et al. “Clindamycin lotion alone versus combination lotion of clindamycin phosphate plus tretinoin versus combination lotion of clindamycin phosphate plus salicylic acid in the topical treatment of mild to moderate acne vulgaris: A randomized control trial”. Indian J Dermatol Venereol Leprol, 2009|Vol 75|Issue 3).

Topical preparations constitute the sole treatment in many patients with acne vulgaris and are a part of therapeutic regimen in almost all patients. As topical treatment, agents containing sulphur or resorcinol were used in the 20th century, salicylic acid was popular for some time, and nowadays retinoids, benzoyl peroxide, azelaic acid, and topical antibiotics are in use (NilFroushzadeh, M. A. et al. “Clindamycin lotion alone versus combination lotion of clindamycin phosphate plus tretinoin versus combination lotion of clindamycin phosphate plus salicylic acid in the topical treatment of mild to moderate acne vulgaris: A randomized control trial”. Indian J Dermatol Venereol Leprol, 2009; Vol 75. Issue 3). However, no single agent is effective in treating all of the major pathophysiological events (Akarsu, S. et al. “Efficacy of the addition of salicylic acid to clindamycin and benzoyl peroxide combination for acne vulgaris”. Journal of Dermatology, 2012; 39: 433-438).

Clindamycin phosphate, an effective and well-tolerated topical antibiotic, has antibacterial and anti-inflammatory effects. Salicylic acid, a topical anti-inflammatory, has keratolytic and comedolytic effects. There have been several clinical studies demonstrating that these two agents alone or in combination are effective in acne lesions (Touitou, E. et al. “Efficacy and tolerability of clindamycin phosphate and salicylic acid gel in the treatment of mild to moderate acne vulgaris”. JEADV, 2008; 22: 616-650). Although standard treatments including antibiotics are commonly used, they become more and more ineffective over time due to the development of resistance to antibiotics. Therefore, alternative treatments including at least one additional active agent were needed. Tea tree oil, an antiseptic, has broad-spectrum antimicrobial and anti-inflammatory effects. The efficacy of tea tree oil in acne treatment has been demonstrated in a clinical study (Enshaieh, S. et al. “The efficacy of 5% topical tea tree oil gel in mild to moderate acne vulgaris: A randomized, double-blind placebo-controlled study”. Indian J Dermatol Venereol Leprol, 2007; 73: 22-5).

Although it has been known in prior art that clindamycin phosphate, salicylic acid and tea tree oil are effective one by one in the treatment of skin infections and acne vulgaris accompanied by inflammatory lesions, there has been no information about the fact that clindamycin phosphate, salicylic acid and tea tree oil are synergistic. Accordingly, it has not been thought that clindamycin phosphate, salicylic acid and tea tree oil can be combined in unit dosage form.

In cases where use of more than one medicine is needed, patient compliance is an important factor to be considered. According to the research, 50% of the patients fail to take medicines as prescribed and to fulfill the requirements of the treatment (Sabaté, E. “Adherence to Long-Term Therapies: Evidence for Action”. World Health Organization. Geneva, 2003. 212 pp.). Patients fail to take their medicines prescribed so as to include a complicated treatment regimen requiring use of more than one medicine due to the facts such as forgetfulness, busy lifestyle, different methods of the administration of the medicines, lack of suitable environment for taking medicines, misunderstanding of the disease or treatment, patient's perspective of the disease or treatment, side effects, depression, cognitive disorders and financial problems. In this case, combining the active components in a single dosage form will increase patient compliance to the treatment.

There has been no information disclosed in prior art relating to the combined use of clindamycin phosphate, salicylic acid and tea tree oil, or combining the active components in a single dosage form. Therefore, there is a need for the topical compositions wherein the patient compliance is increased, wherein active agents are combined in unit dosage form, and wherein said topical compositions are effective in the treatment of skin infections and acne vulgaris accompanied by inflammatory lesions. There is also need for the preparation methods of said topical compositions.

### SUMMARY OF THE INVENTION

The present invention relates to a topical composition comprising clindamycin phosphate, salicylic acid, tea tree oil and at least one topically suitable, pharmaceutically acceptable excipient.

The present invention also relates to the use of a topical composition comprising clindamycin phosphate, salicylic acid, tea tree oil and at least one topically suitable, pharmaceutically acceptable excipient in the treatment of skin infections and acne vulgaris accompanied by inflammatory lesions.

### DETAILED DESCRIPTION OF THE INVENTION

The present invention relates to the topical compositions comprising clindamycin phosphate, salicylic acid and tea tree oil; to the preparation methods thereof; and to the use thereof in the treatment of skin infections and acne vulgaris accompanied by inflammatory lesions.

As a result of the studies, it has been demonstrated that clindamycin phosphate, salicylic acid and tea tree oil included in the topical compositions of the invention are synergistic in the treatment of skin infections and acne vulgaris accompanied by inflammatory lesions.

Synergy between the active agents of the invention in the treatment of skin infections and acne vulgaris accompanied by inflammatory lesions led the present invention directed to combine clindamycin phosphate as a topical antibiotic; salicylic acid as a topical anti-inflammatory; tea tree oil as an antimicrobial and at least one topically suitable pharmaceutically acceptable excipient in unit dosage form.

Topical dosage forms of the invention comprise clindamycin phosphate in an amount of 1%; salicylic acid in an amount of 2%; and tea tree oil in an amount of 2%. It has been determined that pH value of the topical dosage forms of the invention affect the stability. Surprisingly, as a result of the studies, it has been demonstrated that the use of at least one alkalizing/buffering agent, preferably triethanolamine, in a certain amount, preferably in an amount of from 0.5% to 1%, provides the best stability data. Accordingly, optimum pH value of the stable topical dosage forms of the invention is between 6 and 8.

Topical dosage forms of the invention may also include at least one topically suitable pharmaceutically acceptable excipient such as additional alkalizing/buffering agents, emulsifying agents, gelling agents, stiffening agents, rheology modifying/viscosity enhancing agents, surfactants, emollients, humidifiers, preservatives, anti-foaming agents, solvents, topical carriers and smelling agents.

Alkalizing/buffering agents of the invention may include, but are not limited to, ammonia, alkali metal and ammonium hydroxides and carbonates (sodium hydroxide, potassium hydroxide, sodium carbonate, sodium bicarbonate, etc.) and alkanolamines (monoethanolamine, diethanolamine, triethanolamine, monopropanolamine, dipropanolamine, tripropanolamine, etc.).

Emulsifying agents of the invention may include, but are not limited to, cetyl alcohol, cetostearyl alcohol, stearyl alcohol, carboxypolymethylene, polycarbophil, polyethylene glycol, polyvinyl alcohol, polyvinyl acetate, sorbitan esters and amine polymers.

Gelling agents of the invention may include, but are not limited to, carbomers, poloxamers, cellulose derivatives, gelatin, alginic acid, sodium alginate, polyvinyl alcohol and xanthan gum.

Stiffening agents of the invention may include, but are not limited to, cetyl alcohol, cetostearyl alcohol, stearyl alcohol, sorbitan esters and glyceryl esters.

Rheology modifying/viscosity enhancing agents of the invention may include, but are not limited to, carbomers, poloxamers, polymeric gums, hydroxylated fatty acids, fatty esters, fatty amines, fatty waxes, castor oil and derivatives, polycarboxylates, polyacrylates and polymethacrylates.

Surfactants of the invention may include, but are not limited to, sulfates, sulfonates, phosphates, carboxylates, primary-secondary-tertiary amines, quaternary ammonium compounds, fatty alcohols, sugar esters of fatty acids, glycerides of fatty acids, polyoxyethylene glycol alkyl ethers, polysorbates, sorbitan alkyl esters and poloxamers.

Emollients of the invention may include, but are not limited to, white petrolatum (white vaseline), liquid petrolatum (liquid vaseline), paraffin, glycerin and aquaphor.

Humidifiers of the invention may include, but are not limited to, polyhydric alcohols and water-soluble alkoxylated nonionic polymers.

Preservatives of the invention may include, but are not limited to, ethyl alcohol, glycerin, propylene glycol, parabens, phenol, chlorhexidine, chlorocresol, cresol, parahydroxy benzoic acid alkyl esters, benzoic acid and salts, boric acid and salts, citric acid and salts, sorbic acid and salts, neutral preservatives, mercury preservatives and quaternary compounds.

Anti-foaming agent of the invention may include, but is not limited to, dimethicone.

Solvents of the invention may include, but are not limited to, alcohol and water, or any suitable combination thereof.

Topical carriers of the invention may include, but are not limited to, solvents.

Dosage forms of the invention may be in the form of a gel, cream, solution, lotion, ointment or the like for topical administration. Dosage form of the invention is preferably in the form of a gel or solution for topical administration.

Dosage forms of the invention are used in the treatment of skin infections and acne vulgaris accompanied by inflammatory lesions. Dosage forms of the invention may be administered once or twice a day by spreading onto the affected area of the skin as a thin layer after the skin is washed with warm water and dried.

The examples of pharmaceutical formulation and preparation method of the invention are shown below. Said examples are only given to illustrate the invention. The extent of the invention should not be limited by the examples.

### Examples

### Example 1

### Gel Formulation

| Ingredient | Amount (% by weight of the composition) |
|---|---|
| Antibiotic | 1% |
| Anti-inflammatory | 2% |
| Antimicrobial | 2% |
| Gelling agent | 0.5%-2% |
| Anti-foaming agent | 0.5%-5% |
| Alkalizing/Buffering agent | 0.5%-1% |
| Rheology modifying/Viscosity | 15%-50% |
| enhancing agent | |
| Humidifier/Emollient | ≦30% |
| Topical carrier | 5%-50% |
| Smelling agent | 0.1%-0.5% |
| Solvent | q.s.* |

*q.s.: quantity sufficient

### Example 2

### Gel Formulation

| Ingredient | Amount (% by weight of the composition) |
|---|---|
| Clindamycin phosphate | 1% |
| Salicylic acid | 2% |
| Tea tree oil | 2% |
| Carbomer | 2% |
| Dimethicone | 2.5% |
| Triethanolamine | 0.7% |
| Poloxamer | 30% |
| Glycerin | 15% |
| Ethanol | 20% |
| Perfume | 0.3% |
| Distilled water | q.s.* |

*q.s.: quantity sufficient

### Example 3

### Preparation of the Gel Formulation

1. Carbomer is slowly mixed (300-400 rpm) into the distilled water until a homogeneous dispersion is obtained.
2. The obtained mixture is neutralized by the addition of triethanolamine. Mixing rate should be as high as to provide a homogeneous mixture (1000-1200 rpm).
3. Dimethicone is added into the mixture during mixing to prevent foaming (Mixture I).
4. Ethanol and distilled water are mixed to form carrier phase of the gel formulation (Mixture II).
5. Clindamycin phosphate, salicylic acid and tea tree oil in certain amounts are slowly added into Mixture II with moderate stirring (800-1000 rpm) (Mixture III).
6. Poloxamer and glycerin are slowly added into Mixture III with optimum stirring (500-600 rpm).
7. Mixture I and Mixture III are mixed to obtain a homogeneous gel formulation with optimum stirring (300-400 rpm).
8. Perfume is mixed into the final mixture.

### Example 4

### Solution Formulation

| Ingredient | Amount (% by weight of the composition) |
|---|---|
| Antibiotic | 1% |
| Anti-inflammatory | 2% |
| Antimicrobial | 2% |
| Anti-foaming agent | 0.5%-5% |
| Alkalizing/Buffering agent | 0.5%-1% |
| Alkalizing/Buffering agent | q.s.* |
| Preservative | 15%-30% |
| Topical carrier | 5%-50% |
| Smelling agent | 0.1%-0.5% |
| Solvent | q.s.* |

*q.s.: quantity sufficient

### Example 5

### Solution Formulation

| Ingredient | Amount (% by weight of the composition) |
|---|---|
| Clindamycin phosphate | 1% |
| Salicylic acid | 2% |
| Tea tree oil | 2% |
| Dimethicone | 2.5% |
| Triethanolamine | 0.7% |
| Sodium hydroxide | q.s.* |
| Propylene glycol | 20% |
| Isopropyl alcohol | 50% |
| Perfume | 0.3% |
| Distilled water | q.s.* |

*q.s.: quantity sufficient

### Example 6

### Preparation of the Solution Formulation

1. Tea tree oil in a certain amount is mixed (300-400 rpm) into the isopropyl alcohol until the dissolution occurs (Mixture I).
2. Clindamycin phosphate and salicylic acid in certain amounts are slowly added into the distilled water with moderate stirring (800-1000 rpm) (Mixture II).
3. Mixture I and Mixture II are mixed to obtain a homogeneous solution with optimum stirring (300-400 rpm) (Mixture III).
4. Propylene glycol is added into Mixture III during mixing.
5. Dimethicone is added into Mixture III during mixing to prevent foaming.
6. The obtained mixture is neutralized by the addition of triethanolamine. Mixing rate should be as high as to provide a homogeneous mixture (1000-1200 rpm).
7. pH value of the final solution is measured and sodium hydroxide solution is added when needed to adjust the pH to the interval from 6.0 to 8.0.
8. Perfume is mixed into the final mixture.

### Example 7

### Evaluation of the Topical Formulations of the Invention

### 1. Physicochemical Properties

Color, physical appearance, homogeneity and texture properties of the formulations of the invention were determined visually. Said determinations demonstrated that the formulations of the invention are clear and homogeneous, and exhibit uniform texture.

### 2. Spreadability

Spreadability of the formulations of the invention was determined by parallel plate method.

Said determinations demonstrated that the formulations of the invention are easily spread by applying little force.

3. pH

pH value of the formulations of the invention was determined by using digital pH meter. pH interval of the formulations of the invention is from 6 to 8. Because said pH interval is consistent with normal pH of the skin, skin irritation is not expected.

### 4. Content Uniformity

Content uniformity of the formulations of the invention was determined by applying conventional quantitative assay to the samples taken from upper sample point, mid sample point and lower sample point. Said determinations demonstrated that content of the formulations of the invention is uniform.

### 5. Stability

Stability of the formulations of the invention was determined by long term stability studies performed at 25±2° C. and 60±5% RH across a 3- and 6-month follow-up period; and accelerated stability studies performed at 40±2° C. and 75±5% RH across a 3-month follow-up period. Said determinations demonstrated that there is not any significant difference in the pH and other physical properties of the formulations of the invention depending on temperature, humidity and time.

1. Topical composition comprising clindamycin phosphate, salicylic acid, tea tree oil and at least one topically suitable, pharmaceutically acceptable excipient.

2. Topical composition according to claim 1, wherein said composition comprises clindamycin phosphate in an amount of 1%.

3. Topical composition according to claim 1, wherein said composition comprises salicylic acid in an amount of 2%.

4. Topical composition according to claim 1, wherein said composition comprises tea tree oil in an amount of 2%.

5. Topical composition according to claim 1, wherein said composition comprises alkalizing/buffering agent as at least one topically suitable, pharmaceutically acceptable excipient.

6. Topical composition according to claim 5, wherein said composition comprises triethanolamine as alkalizing/buffering agent.

7. Topical composition according to claim 6, wherein said composition comprises triethanolamine in an amount of from 0.5% to 1%.

8. Topical composition according to any one of the claims 1 to 7, wherein said composition is in the form of gel.

9. Gel composition according to claim 8, wherein said composition comprises gelling agent as at least one topically suitable, pharmaceutically acceptable excipient.

10. Gel composition according to claim 9, wherein said composition comprises carbomer as gelling agent.

11. Gel composition according to claim 10, wherein said composition comprises carbomer in an amount of from 0.5% to 2%.

12. Gel composition according to claim 8, wherein said composition comprises rheology modifying/viscosity enhancing agent as at least one topically suitable, pharmaceutically acceptable excipient.

13. Gel composition according to claim 12, wherein said composition comprises poloxamer as rheology modifying/viscosity enhancing agent.

14. Gel composition according to claim 13, wherein said composition comprises poloxamer in an amount of from 15% to 50%.

15. Gel composition according to claim 8, wherein said composition comprises humidifier/emollient as at least one topically suitable, pharmaceutically acceptable excipient.

16. Gel composition according to claim 15, wherein said composition comprises glycerin as humidifier/emollient.

17. Gel composition according to claim 16, wherein said composition comprises glycerin in an amount up to 30%.

18. Gel composition according to claim 8, wherein said composition comprises anti-foaming agent as at least one topically suitable, pharmaceutically acceptable excipient.

19. Gel composition according to claim 18, wherein said composition comprises dimethicone as anti-foaming agent.

20. Gel composition according to claim 19, wherein said composition comprises dimethicone in an amount of from 0.5% to 5%.

21. Gel composition according to claim 8, wherein said composition comprises solvent as at least one topically suitable, pharmaceutically acceptable excipient.

22. Gel composition according to claim 21, wherein said composition comprises distilled water as solvent.

23. Gel composition according to claim 8, wherein said composition comprises topical carrier as at least one topically suitable, pharmaceutically acceptable excipient.

24. Gel composition according to claim 23, wherein said composition comprises ethanol as topical carrier.

25. Gel composition according to claim 24, wherein said composition comprises ethanol in an amount of from 5% to 50%.

26. Gel composition according to claim 8, wherein said composition optionally comprises at least one topically suitable, pharmaceutically acceptable excipient selected from a group comprising emulsifier, stiffening agent, surfactant, preservative and smelling agent.

27. Gel composition according to claim 8, wherein said composition comprises:

| | |
|---|---|
| Clindamycin phosphate | 1% |
| Salicylic acid | 2% |
| Tea tree oil | 2% |
| Carbomer | 2% |
| Dimethicone | 2.5% |
| Triethanolamine | 0.7% |
| Poloxamer | 30% |
| Glycerin | 15% |
| Ethanol | 20% |
| Perfume | 0.3% |
| Distilled water | q.s. |

28. Preparation method of the gel composition according to claim 27, wherein said method comprises the steps of:
a. mixing carbomer into the distilled water slowly until a homogeneous dispersion is obtained,
b. neutralizing the obtained mixture by the addition of triethanolamine and mixing until a homogeneous mixture is obtained,
c. adding dimethicone into the mixture during mixing to prevent foaming and obtaining Mixture I,
d. mixing ethanol and distilled water to form carrier phase of the gel formulation and obtaining Mixture II,
e. adding clindamycin phosphate, salicylic acid and tea tree oil into Mixture II slowly with moderate stirring and obtaining Mixture III,
f. adding poloxamer and glycerin into Mixture III slowly with optimum stirring,
g. mixing Mixture I and Mixture III to obtain a homogeneous gel formulation with optimum stirring, and
h. mixing perfume into the final mixture.

29. Topical composition according to any one of the claims 1 to 7, wherein said composition is in the form of solution.

30. Solution composition according to claim 29, wherein said composition comprises anti-foaming agent as at least one topically suitable, pharmaceutically acceptable excipient.

31. Solution composition according to claim 30, wherein said composition comprises dimethicone as anti-foaming agent.

32. Solution composition according to claim 31, wherein said composition comprises dimethicone in an amount of from 0.5% to 5%.

33. Solution composition according to claim 29, wherein said composition comprises preservative as at least one topically suitable, pharmaceutically acceptable excipient.

34. Solution composition according to claim 33, wherein said composition comprises propylene glycol as preservative.

35. Solution composition according to claim 34, wherein said composition comprises propylene glycol in an amount of from 15% to 30%.

36. Solution composition according to claim 29, wherein said composition comprises solvent as at least one topically suitable, pharmaceutically acceptable excipient.

37. Solution composition according to claim 36, wherein said composition comprises distilled water as solvent.

38. Solution composition according to claim 29, wherein said composition comprises topical carrier as at least one topically suitable, pharmaceutically acceptable excipient.

39. Solution composition according to claim 38, wherein said composition comprises isopropyl alcohol as topical carrier.

40. Solution composition according to claim 39, wherein said composition comprises isopropyl alcohol in an amount of from 5% to 50%.

41. Solution composition according to claim 29, wherein said composition optionally comprises at least one topically suitable, pharmaceutically acceptable excipient selected from a group comprising alkalizing/buffering agent, emulsifier, stiffening agent, rheology modifying/viscosity enhancing agent, surfactant, humidifier/emollient and smelling agent.

42. Solution composition according to claim 29, wherein said composition comprises:

| | |
|---|---|
| Clindamycin phosphate | 1% |
| Salicylic acid | 2% |
| Tea tree oil | 2% |
| Dimethicone | 2.5% |
| Triethanolamine | 0.7% |
| Sodium hydroxide | q.s. |
| Propylene glycol | 20% |
| Isopropyl alcohol | 50% |
| Perfume | 0.3% |
| Distilled water | q.s. |

43. Preparation method of the solution composition according to claim 42, wherein said method comprises the steps of:
a. mixing tea tree oil into the isopropyl alcohol until the dissolution occurs and obtaining Mixture I,
b. adding clindamycin phosphate and salicylic acid into the distilled water slowly with moderate stirring and obtaining Mixture II,
c. mixing Mixture I and Mixture II to obtain a homogeneous solution with optimum stirring and obtaining Mixture III,
d. adding propylene glycol into Mixture III during mixing,
e. adding dimethicone into Mixture III during mixing to prevent foaming,
f. neutralizing the obtained mixture by the addition of triethanolamine and mixing until a homogeneous mixture is obtained,
g. adding sodium hydroxide solution to adjust the pH value of the final solution to the interval from 6.0 to 8.0,
h. mixing perfume into the final mixture.

44. Use of a topical composition according to any one of the preceding claims in the treatment of acne vulgaris.

45. Use of a topical composition according to any one of the preceding claims in the treatment of skin infections.

46. Use of a topical composition according to any one of the preceding claims once or twice a day.

47. Use of a topical composition comprising clindamycin phosphate, salicylic acid, tea tree oil and at least one topically suitable, pharmaceutically acceptable excipient in the treatment of acne vulgaris.

48. Use of a topical composition comprising clindamycin phosphate, salicylic acid, tea tree oil and at least one topically suitable, pharmaceutically acceptable excipient in the treatment of skin infections.
